(19) United States
(12) Patent Application Publication
(10) Pub. No.: US 2009/0149423 A1
(43) Pub. Date: Jun. 11, 2009

(54) USE FOR A VERY LOW MOLECULAR WEIGHT HEPARINS

(75) Inventors: Pietro Bianchini, Modena (IT); Giuseppe Mascellani, Monte S. Pietro (IT); Bruna Parma, Modena (IT)

Correspondence Address: MCCORMICK, PAULDING & HUBER LLP, CITY PLACE II, 185 ASYLUM STREET, HARTFORD, CT, 06103, US

(73) Assignee: OPOCRIN S.P.A., Corlo Di Formigine (IT)

(21) Appl. No.: 11/718,191

(22) PCT Filed: Oct. 27, 2005
(86) PCT No.: PCT/EP05/55593

§ 371 Date: Apr. 27, 2007

(30) Foreign Application Priority Data
Oct. 29, 2004 (IT) MI2004A002068

(51) Int. Cl. A61K 31/727; A61P 19/10

(52) U.S. Cl. 514/56

(57) ABSTRACT

The present invention relates to the use of heparin oligosaccharides having a molecular weight≦5050 Da and an anti-factor Xa activity≦80 IU/mg, or their sodium salts, for preparing a medicament for the prevention and treatment of osteoporosis and associated pathologies and for preparing a medicament effective for the prevention and treatment of cerebral ischemia and pathologies related thereto whose treatment necessitates drugs able to cross the blood-brain barrier.

### STATE OF THE ART

So-called unfractionated heparin (UFH) has an average molecular weight of about 12000-15000 Da and has primarily anti-coagulant properties. Treatment with heparin presents a number of side effects which considerably limit the possibility of utilizing this substance, the most substantial of these being the onset of hemorrhagic and osteoporotic phenomena.

In particular, unfractionated heparin presents the drawback that a third of patients treated for longer than three months develop an asymptomatic reduction in bone density with onset of osteoporosis and 2-3% of patients develop bone fractures (Barbour L. A. et al., Am. J. Obst. Gynecol. 170, 862, 1994; Dahlman T. C., Am. J. Obst. Gynec., 168, 1265, 1993).

In recent years research has been directed towards developing low molecular weight heparins with fewer side-effects.

Low molecular weight heparins (LMWH) are produced by chemical or enzymatic depolymerization of unfractionated heparin molecules and have a molecular weight less than 8000 Da. Because of the various fragmentation and/or fractionation procedures used, low molecular weight heparins constitute a heterogeneous class of compounds that differ in molecular weight, pharmacokinetics and activity. Low molecular weight heparin is universally used in all pathologies, particularly thromboembolic pathologies, whose prevention and treatment can be mediated by the inhibition of thrombin by anti-thrombin III (AT III).

These compounds possess mainly antithrombotic properties and therefore the resulting hemorrhagic-type side effects are very few.

However, numerous clinical experiments and retrospective investigations, based on observations following their entry onto the market, have indicated that even low molecular weight heparins cause a significant incidence of bone fractures in patients undergoing treatment, though less notable than observed with unfractionated heparin (Monreal, M. et al, Thromb. Haemost., 71, 7, 1994).

The mechanism at the basis of the aforesaid phenomena has proved to be the capacity of heparin itself to inhibit (even at a concentration of 5 μg/ml) the proliferation of osteoblasts in culture, formation of bone matrix and its mineralization.

Studies on experimental animals indicate that the effect on bone density is dependent on the chain length, hence on molecular weight, and on the degree of sulfation of heparin, being less marked with decreasing molecular weight of the heparin fractions used (Hirsh J., Circulation 98, 1575-1582, 1998; Shaughnessy S. G. et al., Blood 86, (4), 1368-1373, 1995).

The present inventors have described in U.S. Pat. No. 4,791,195, U.S. Pat. No. 4,933,326 and EP 0439555 specific ultra low molecular weight heparins, obtained by the peroxy-radical depolymerization of heparin, and which possess anti-thrombotic and/or anti-arteriosclerotic activity. These oligosaccharides, of average molecular weight less than that of LMWHs, have a different mechanism of action than traditional low molecular weight heparins, presenting very little affinity for antithrombin III and having an anti-factor Xa activity≦80 IU/mg.

### SUMMARY

The present inventors have now found that heparin fractions having a molecular weight≦5050 Da and having an anti-factor Xa activity≦80 IU/mg, preferably including the very low molecular weight heparins described in U.S. Pat. No. 4,791,195, U.S. Pat. No. 4,933,326 and EP 0439555 in the name of the present applicant, possess unexpected pharmacological properties that are not observed with other low molecular weight heparins having anti-factor Xa activity higher than the aforesaid value.

Indeed, these oligosaccharides are not only free of the pro-osteoporotic activity observed with unfractionated heparin or other low molecular weight heparins, but surprisingly demonstrate in comparison a reversal of activity on bone mass. Specifically, these heparin fractions are able to modulate intra- and extra-cellular calcium movements and to act as specific inhibitors of osteoclastic bone resorption. These compounds therefore exhibit an inhibitory activity on osteoporosis onset and, by virtue of their aptitude for inhibiting calcium salt crystallization, simultaneously act as inhibitors of calcium depositing in tissues and arterial and venous vessels.

The present invention thus relates to the use of the aforesaid very low molecular weight heparins for preparing medicaments for the prevention and treatment of osteoporosis, calcification of arterial and venous vessels and pathologies that result from calcium deposits in tissues, for example urolithiasis.

Furthermore, the present inventors have surprisingly found that the aforesaid oligosaccharides also have a capacity to reduce brain damage induced by cerebral ischemia to far greater effect than low molecular weight heparins (LMWH) possessing higher anti-Xa activity values.

The present invention therefore also relates to the use of the aforesaid very low molecular weight heparins for preparing a medicament for the prevention and treatment of the damage following a cerebral ischemia.

### DESCRIPTION OF THE FIGURES

FIG. 1.1 shows the percentage of calcium, relative to aorta weight, accumulated in the aortic tissue of normal rats (NC) or rats rendered arteriosclerotic and treated with placebo (PC) or with 15 mg/kg of Deligoparin by oral means (Deligoparin).

FIG. 1.2 shows the percentage of calcium accumulated in the cardiac tissue of normal rats (NC) or rats rendered arteriosclerotic and treated with placebo (PC), or with 17 mg/kg of Deligoparin by oral means (Deligoparin).

FIG. 2 shows the extent, expressed in percentage, of inflammatory effect (edema-white histograms) and cerebral infarct/ischemia (TTC dicoloration—percent variation of immunohistochemical staining by triphenyltetrazolium chloride (TTC)-black histograms) in operated senescent rats, as control, in the group of rats treated sub-cutaneously (sc) with 2.5 mg/kg of enoxaparin and in the group of rats treated sub-cutaneously (sc) with 2.5 mg/kg of Deligoparin (Oligo H).

### DETAILED DESCRIPTION OF THE INVENTION

The present inventors have found that heparin oligosaccharides having molecular weight less than 5050 Da and an anti-factor Xa activity≦80 IU/mg, and their sodium salts, are able to inhibit osteoclastic bone resorption and to regulate intra- and extra-cellular calcium transport. Therefore, the aforesaid oligosaccharides are able on the one hand to inhibit the onset and progression of osteoporosis and on the other hand to inhibit mineralization (calcification) of tissues and arterial and venous vessels.

Hence a first aspect of the present invention is the use of the aforesaid oligosaccharides for preparing a medicament for the prevention and treatment of osteoporosis.

The present invention also relates to the use of the aforesaid oligosaccharides for preparing a medicament for the prevention and treatment of arterial and venous vessel calcification and pathologies resulting from calcium deposits in tissues, preferably to include urolithiasis.

The present inventors have also found that, as will be demonstrated in Example 2 to follow, the aforesaid oligosaccharides show a far greater effectiveness in inhibiting and reducing the damage derived from cerebral ischemia than that shown by conventional low molecular weight heparins (LMW-H).

Therefore, a further aspect of the present invention is the use of the aforesaid oligosaccharides for the prevention and treatment of damage following a cerebral ischemia.

The oligosaccharides of the present invention are obtained by the depolymerization of heparin, preferably of porcine or bovine origin. The methods for the characterisation and analysis of heparin fractions and fragments are those described in the European Pharmacopeia monograph relating to low molecular weight heparin.

Particularly preferred heparin oligosaccharides in accordance with the present invention are those described in U.S. Pat. No. 4,933,326, U.S. Pat. No. 4,791,195 and EP 439555, in the name of the proprietor, and which are incorporated herein by reference.

In detail, the preferred oligosaccharides for use in the present invention are chosen from the group consisting of:

a) a mixture consisting of heparin oligosaccharides containing an average of 13-17 saccharide units and having average molecular weights between 3580 and 5050 Da, preferably of about 3580 Da, 4000 Da, 4570 Da, 4580 Da and 5050 Da; b) heparin oligosaccharides containing an average of 4-12 saccharide units and having average molecular weights between 1000 Da and 3570 Da, preferably of about 1000 Da, 1700 Da, 2000 Da, 2100 Da, 2300 Da, 2880 Da, 3200 Da, 3500 Da or 3570 Da;

the aforesaid oligosaccharides containing terminal monosaccharides consisting of glucosamine N,6-disulfate and iduronic acid 2-sulfate, in which the anomeric carbon is reducing.

All the aforesaid average molecular weights are considered to have a confidence interval that depends on the limitations of the methods for molecular weight determination as described in the Pharmacopeias.

Of the aforesaid heparin oligosaccharides, particularly preferred is Deligoparin (INN), obtained by the depolymerization of porcine heparin and having an average molecular weight of about 3200 Da.

As will be shown in Example 1 to follow, Deligoparin is able to inhibit calcification of the arteries in a similar manner to osteoprotegerin (Price A. P. et al. Arterioscl. Thromb. Vasc. Biol. 21 1610-1616, 2001).

Furthermore, as will be shown in Example 2 to follow, the present inventors have also found that Deligoparin is able to reduce by 52% the damage resulting from cerebral ischemia compared to the positive control, while enoxaparin, having an intrinsic anti-factor Xa activity of about 110 IU/mg, i.e. double that of Deligoparin, is able to reduce said damage by only 21%.

Therefore, a further aspect of the present invention is the use of the aforesaid oligosaccharides for the prevention and treatment of the damage following a cerebral ischemia.

The oligosaccharides of the invention are effective not only when administered by injection but also by oral means and by intrabronchial inhalation. In man, the oligosaccharides are utilized at a daily dosage between 0.5 and 10 mg/kg, depending on the administration means employed.

The present invention will now be better illustrated by the following examples.

### EXAMPLE 1

### Inhibition of Calcium Deposits by Deligoparin in the Aorta and Heart of Rats

The heparin oligosaccharide Deligoparin (Oligo H), having an average molecular weight of 3200 Da and an anti-Xa activity of about 57 IU/mg, was studied in a model of arteriosclerotic rats (the Villaverde model, described in Padrò T. et. al., Thromb. Res. 49, 519-530, 1988; Villaverde C. A., Drugs of Today 24 (S1), 79, 1988).

10 rats were rendered arteriosclerotic by treating them with a mixture of vitamin D2 and cholesterol dissolved in olive oil for 3 consecutive days and then with a diet of fats, sugars and proteins for 18 days.

A second group of 10 rats received no treatment and was used as the control (normal rats).

Other groups each of 10 rats also received 15 mg/kg of Deligoparin orally or subcutaneously or 17 mg/kg orally after the third day and for 18 consecutive days, in addition to the aforesaid diet.

The rats which were rendered arteriosclerotic displayed very obvious calcifications of the aortic endothelium, also clearly visible on the exterior of the arteries which appeared hard and deformed. Large calcified plaques were evident at the arch and were particularly numerous at the points where the arteries emerge from the arch itself. Portions of the thoracic and abdominal aortas also displayed very evident plaques. The adventitia of the aorta was coated with a very sticky whitish fat, also the presence of vasa vasorum was evident in much greater numbers than with the aorta of the control group. The hearts and kidneys were considerably smaller than those of the control group whilst the livers were very yellow in colour with clear signs of steatosis.

The rats treated with Deligoparin, particularly those treated orally, displayed aortas that were far more elastic and virtually uncalcified, being less infiltrated by fat and vasa vasorum.

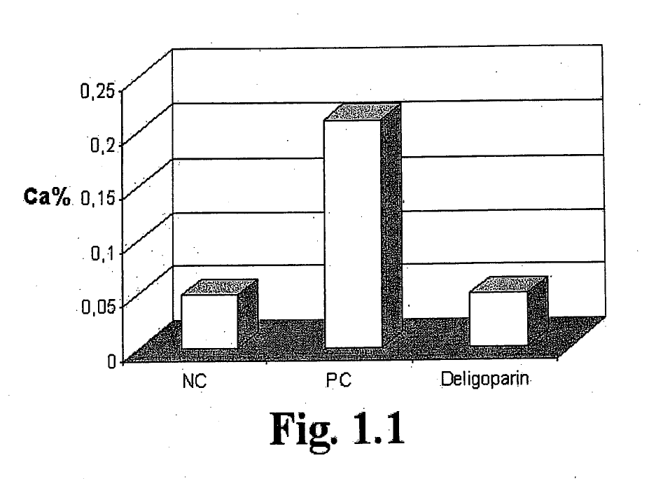

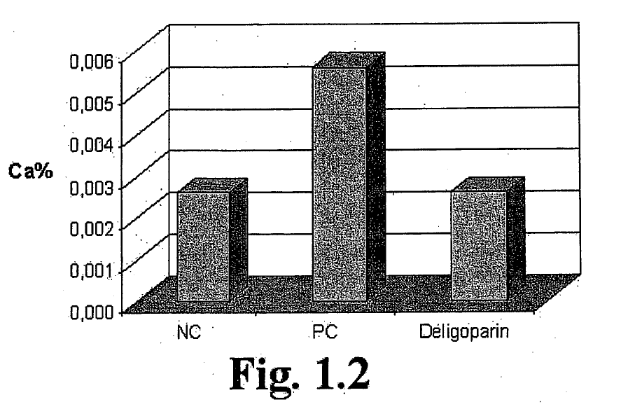

The calcium content (analysed by atomic absorption) in the aortas of the three groups of rats (normal, rendered arteriosclerotic and treated with 15 mg/kg of Oligo H by mouth) is given in the histogram shown in FIG. 1.1. As an example, in FIG. 1.2 the calcium content of the hearts of rats treated orally with 17 mg/kg of Oligo H is given.

### EXAMPLE 2

### Inhibition of Damage Induced by Cerebral Ischemia in Rats

Groups of 7-10 senescent male Wistar rats weighing 300-350 g were utilized.

A focal ischemia was produced surgically by partial occlusion of the middle cerebral artery, in accordance with Derugin's method (Olah L., Wecker S. and Hoehn M., Journal of Cerebral Blood Flow and Metabolism 20 (10), 1474-82, 2000; Derugin et al. Stroke 31 (7) 1752-1761, 2000).

6 hours following the pro-thrombotic occlusion, various groups of rats were treated separately with 2.5 mg/kg doses of Oligo H (Deligoparin) or enoxaparin intravenously or subcutaneously twice a day for a period of one week.

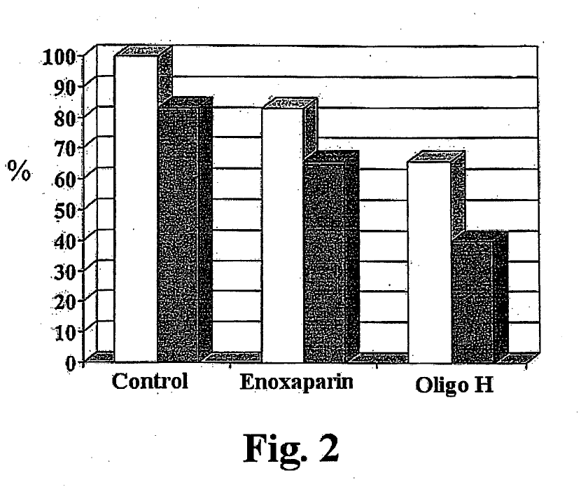

The damage from the cerebral infarct/ischemia was identified and quantified by computer analysis of images of brain sections stained with tetrazolium salts. FIG. 2 shows the histograms relating to the subcutaneously administered 2.5 mg/kg dose.

The effect is surprising in that the LMWH enoxaparin (INN), characterised by an intrinsic activity, expressed in IUs of one tenth activated anti-factor (aXa), which is double that of Deligoparin, has provided a result of about one half. At doses of 2.5 mg/kg (140 IU aXa/kg, twice a day for one week), the heparin fragment of molecular weight 3200 Da is able to inhibit stroke by 52% while the enoxaparin of about 4500 Da, at doses of about 270 IU aXa/kg twice a day for one week, inhibits stroke by only 21%.

1. Method for preventing and treating osteoporosis, calcification of arterial and venous vessels and pathologies resulting from calcium deposits in tissues, comprising administering a heparin oligosaccharide having a molecular weight less than 5050 Da and an anti-factor Xa activity<80 IU/mg, or the sodium salts, to a patient in need thereof.

2. Method as claimed in claim 1, wherein said pathology resulting from calcium deposits in tissues is urolithiasis.

3. Method as claimed in claim 1, wherein said oligosaccharides are chosen from the group consisting of:
a) a mixture consisting of heparin oligosaccharides containing an average of 13-17 saccharide units and having average molecular weights between 3580 and 5050 Da; and
b) heparin oligosaccharides containing an average of 4-12 saccharide units and having average molecular weights between 1000 Da and 2570 Da;
the aforesaid oligosaccharides containing terminal monosaccharides consisting of glucosamine, N,6-disulfate and iduronic acid 2-sulfate, in which the anomeric carbon is reducing.

4. Method as claimed in claim 3, wherein the oligosaccharides a) consist of a mixture of oligosaccharides having an average molecular weight of about 3580 Da, 4000 Da, 4570 Da, 4580 Da and 5050 Da.

5. Method as claimed in claim 3, wherein the oligosaccharides b) have an average molecular weight of about 1000 Da, 1700 Da, 2000 Da, 2100 Da, 2300 Da, 2880 Da, 3200 Da, 3500 Da or 3570 Da.

6. Method as claimed in claim 5, wherein said oligosaccharides have an average molecular weight of about 3200 Da.

7. Method for preventing and treating damages following a cerebral ischemia, comprising administering a heparin oligosaccharide having a molecular weight less than 5050 Da and an anti-factor Xa activity<80 IU/mg, or the sodium salts, to a patient in need thereof.

8. Method as claimed in claim 7, wherein said oligosaccharides are chosen from the group consisting of:
a) a mixture consisting of heparin oligosaccharides containing an average of 13-17 saccharide units and having average molecular weights between 3580 and 5050 Da;
b) heparin oligosaccharides containing an average of 4-12 saccharide units and having average molecular weights between 1000 Da and 3570 Da;
the aforesaid oligosaccharides containing terminal monosaccharides consisting of glucosamine N,6-disulfate and iduronic acid 2-sulfate, in which the anomeric carbon is reducing.

9. Method as claimed in claim 8, wherein the oligosaccharides a) consist of a mixture of oligosaccharides having an average molecular weight of about 3580 Da, 4000 Da, 4570 Da, 4580 Da and 5050 Da.

10. Method as claimed in claim 8, wherein the oligosaccharides b) have an average molecular weight of about 1000 Da, 1700 Da, 2000 Da, 2100 Da, 2300 Da, 2880 Da, 3200 Da, 3500 Da or 3570 Da.

11. Method as claimed in claim 10, wherein said oligosaccharides have an average molecular weight of about 3200 Da.

12. Method as claimed in claim 1, wherein said medicament is suitable for oral administration.
